(19) United States
(12) Patent Application Publication
(10) Pub. No.: US 2003/0045497 A1
(43) Pub. Date: Mar. 6, 2003

(54) Methylation resistant vectors

(75) Inventors: Bengt Widegren, Lund (SE); Bertil Persson, Lund (SE); Leif G. Salford, Lund (SE)

Correspondence Address: David D. Stein, BOYLE, FREDRICKSON, NEWHOLM, STEIN & GRATZ, S.C., 250 Plaza, Suite 1030, 250 East Wisconsin Avenue, Milwaukee, WI, 53202, US

(73) Assignee: Geneinvent BBL AB

(21) Appl. No.: 10/206,557

(22) Filed: Jul. 26, 2002

Related U.S. Application Data
(60) Provisional application No. 60/308,549, filed on Jul. 27, 2001.

(51) Int. Cl. A61K 48/00; C12N 15/74

(52) U.S. Cl. 514/44; 424/93.2; 435/320.1

(57) ABSTRACT

The invention relates to vectors produced in a donor host cell, which upon transfer into a receiver host cell maintain the desired expression of the nucleotide sequences that are located within the vector. The maintenance of the desired expression is achieved because the vector at least partly remains unmethylated within the receiver host cell. The donor host cell is different as compared to the receiver host cell and the receiver host cell being capable of methylating DNA. The invention also relates to methods for the production of such vectors and the use of the vectors in industry as well as in medicine.

### CROSS-REFERENCE TO RELATED APPLICATION

This application claims priority under 35 U.S.C. § 119(e) to U.S. Provisional Patent Application No. 60/308,549, filed Jul. 27, 2001, the entirety of which is incorporated by reference herein.

### FIELD OF THE INVENTION

The invention relates to vectors, which at least partly remains resistant to methylation upon transfer into a receiver host cell. The receiver host cell is different from the donor host cell. The invention also relates to methods for the production of such vectors and the use of the vectors in industry as well as in medicine.

### BACKGROUND OF THE INVENTION

Modulation of gene expression has become an increasingly important approach to understand various cellular processes and their underlying biochemical pathways. Regulation of gene expression is a complex process, and many mechanisms of regulation remain to be understood. Methylation of DNA in eukaryotic organisms is one important mechanism of gene regulation. Methylation of DNA is a mechanism to control the expression of genes located within the genome of eukaryotic cells as well as to downregulate the expression of foreign nucleotide sequences that enters into a eukaryotic cell. The phenomenon has been investigated in both animal cells as well as in plants, and the methylation is identified to occur on the 5-position in the pyrimidine ring of cytosine located in a stretch or sequence of CpG.

Silencing of a gene or a cluster of genes may be the result of methylation of CpG motifs located in the nucleotide sequence of the gene or cluster of genes, as well as methylation of regulatory sequences, such as promoters and responsive elements for transcription factors. The degree of methylation influences the degree of silencing and, in general, a low degree of methylation results in a minor decrease in expression, and a higher degree of methylation results in complete silencing of the expression of a gene.

Methylation affects the expression of polypeptides located within expression vectors. When a stretch or sequence of foreign DNA enters the nucleus of a cell, it is likely that the stretch of foreign DNA become methylated. The degree of methylation of a sequence of foreign DNA will affect the level of expression from that particular DNA. The introduced DNA can also be integrated into the chromosomal DNA of a dividing cell by recombination. If that particular DNA becomes methylated prior to integration into the chromosomal DNA and cell division, it will probably become methylated during subsequent cell divisions. If a high level of expression is desired, then methylation of the DNA in expression vectors is a problem.

A similar problem is also found when vectors are used in medicine for treatment of disorders and for vaccination. For example, a DNA vaccine is most often presented in the form of a plasmid DNA expression vector produced in bacteria and then purified before delivering it, such as by injection, to muscle or skin tissue. DNA vaccines have been shown efficacy against numerous viral, bacterial, and parasitic disorders or infections in animal models. However, the level of methylation of such DNA will affect the level of expression of antigenic proteins essential for the induction of an immune response against the antigen. The same problem also applies to the use of DNA vectors in gene therapy.

One way to solve the methylation problem of nucleotide sequences upon transfer into a new host is by manipulation of the genes in order to remove all CpG motifs without changing the encoded polypeptides. Such modified genes will not undergo methylation. However, the strategy of using such modified genes is well suited for genes with a shorter nucleotide sequence, but not as applicable for larger nucleotide sequences, such as vectors or when there is a need for several different vectors. Moreover, promoters used for the expression of genes are often rich in CpG motifs, which cannot be removed without impairing the function of the promoter and thereby the expression level.

Thus, there is a need for stable optimised vectors, which do not undergo methylation upon transfer into a eukaryotic cell for use in both industry and medicine. By provision of such vectors, the industry will have lesser problems in obtaining constant and higher expression of genes of interest in eukaryotic cells.

There also is a need in medicine to provide improved vectors that do not undergo methylation upon transfer into a receiver host cell and the expression of the nucleotide sequences located within the vectors are maintained, which is useful both in vaccination and in gene therapy, as well as all in other treatments in which expression vectors are used.

### SUMMARY OF THE INVENTION

The invention relates to vectors which have been modified in such a way that methylation of the vector is substantially reduced upon transfer of the vector into a receiver host cell. As a result, expression of the nucleotide sequence of the vector within a receiver host cell is maintained at an acceptable level. An example of an acceptable level is that which maintains the expected functions of the nucleotide sequences and/or the polypeptides encoded by the nucleotide sequences. The invention also relates to methods for the production of such vectors and the use of the vectors in industry as well as in pharmaceuticals, e.g., therapy and/or diagnostics.

Accordingly, in a first aspect, the invention relates to a vector comprising a nucleotide sequence, where one or two cytosines in at least one CpG motif has been replaced with a cytosine analogue that is resistant to methylation.

In another aspect, the invention relates to a donor host cell comprising a vector with a nucleotide sequence, where one or two cytosines in at least one CpG motif has been replaced with a cytosine analogue resistant to methylation. hi a further aspect, the invention relates to a nucleotide sequence being part of a vector obtained from a donor host cell and/or a polypeptide produced by a donor host cell.

In a still further aspect, the invention relates to a pharmaceutical composition comprising a vector and/or a donor host cell and/or a nucleotide sequence and/or a polypeptide and a pharmaceutically acceptable diluent, carrier, adjuvant or excipient.

In still another aspect, the invention relates to a method of reducing methylation of CpG motifs in a vector in a receiver host, which comprises replacing at least one cytosine in a CpG motif with a cytosine analogue.

In an additional aspect, the invention relates to a kit comprising a vector and/or a donor host cell.

In a still further aspect, the invention relates to a method of transferring a vector, a nucleotide sequence, a polypeptide or pharmaceutical composition into a receiver host cell, the method being selected from a list consisting of electroporation, microprojectile bombardment and liposome mediated delivery.

In still another aspect, the invention relates to a vector, a donor host cell, a nucleotide sequence, a polypeptide, or a pharmaceutical composition for use in therapy, diagnostics, or both.

In perhaps a final aspect, the invention relates to the use of a vector, a donor host cell, a nucleotide sequence, a polypeptide or a pharmaceutical composition for the manufacture of a medicament for use in therapy, diagnostics, or both.

The invention provides completely novel vectors in which cytosine in CpG motifs have been replaced with a cytosine analogue. The cytosine analogue preferably is resistant against methylation. The cytosine analogue replaces at least the 5-position of the pyrimidine ring of cytosine in the CpG motif. Such a replacement results in an improved vector, which upon transfer into a receiver host cell, such as an eukaryotic cell, helps maintain the activity/expression of the nucleotide sequence of the vector. The invention further provides one or more methods for the production of such vectors and the use of such vectors in, e.g., industry, as well as in medicine.

Other objects, features, and advantages of the present invention will become apparent to those skilled in the art from the detailed description and the accompanying drawings. It should be understood, however, that the detailed description and accompanying drawings, while indicating one or more preferred embodiments of the present invention, are given by way of illustration and not of limitation. Many changes and modifications may be made within the scope of the present invention without departing from the spirit thereof, and the invention includes all such modifications.

### BRIEF DESCRIPTION OF THE DRAWINGS

These and other objects, features, and advantages of this invention will become apparent from the following detailed description of the best mode, appended claims, and accompanying drawings in which:

FIG. 1 shows a structure of 5-azadeoxycytidine; and

FIG. 2 shows an example of a general structure of a cytidine derivative.

### DETAILED DESCRIPTION OF AT LEAST ONE PREFERRED EMBODIMENT

### Definitions

In the context of the present application and invention, the following definitions apply:

The term “nucleotide sequence” is intended to mean a sequence of two or more nucleotides. The nucleotides may be of genomic DNA, CDNA, RNA, semisynthetic or synthetic origin or a mixture thereof. The term includes circular, linear, single and double stranded forms of DNA or RNA.

The term “vector” is intended to mean a nucleotide sequence, usually being a circular duplex of DNA having the ability to multiply independently of chromosomal DNA into numbers of copies in a host, i.e., having a start of replication. The vector may also be a vector that integrates into the genome of the receiver host cell. Furthermore the vector is stably maintained and/or produced in the host during multiplication, such as by the use of a selectable marker in the vector. The vector may be a bacteriophage, a plasmid, a phagemid, a viral vector, a plant transformation vector, an insect vector, or a yeast artificial chromosome. Additionally, the vector may be a nucleotide sequence useful as antisense or to form aptameres.

The term “start of replication” is intended to mean a nucleotide sequence at which DNA synthesis for replication of the vector begins. Start of replication may occur at one or more points within the vector dependent on the vector being used, such as at one point in a plasmid vector or at several points in an adenovector. The start of replication is generally termed origin of replication (abbreviated ori site) in a plasmid vector.

The term “selectable marker” is intended to mean, e.g., a gene encoding a polypeptide that confers resistance to a drug, e.g., ampicillin, kanamycin, tetracyclin, chloramphenicol, neomycin, hygromycin, or methotrexate.

The term “control sequence” or “control sequences” is intended to mean nucleotide sequences involved in control of a response to an action. This includes nucleotide sequences and/or proteins involved in regulating, controlling, or affecting the expression of structural genes, or the replication, selection, or maintenance of a plasmid or a viral vector. Examples include attenuators, silencers, enhancers, operators, terminators, and promoters.

The term “CpG motif” is intended to mean a double-stranded nucleotide sequence having a cytosine followed by a guanine linked by a phosphate bond. A CpG motif has two cytosines with one cytosine located in each strand of the double-stranded nucleotide sequence.

The term “methylation” or “methylated CpG motif” is intended to mean that cytosine is methylated on the pyrimidine ring, which usually occurs at the 5-position of the pyrimidine ring. One or more methylated CpG motifs in a nucleotide sequence can cause complete silencing of the expression of the nucleotide sequence. If the methylated CpG motifs are located (1) within a nucleotide sequence encoding a polypeptide, or (2) within other nucleotide sequences involved in the expression of a specific nucleotide sequence, e.g., a control sequence as defined above, substantial or complete reduction of the expression of the nucleotide sequence is obtained.

The term “resistant against methylation” is intended to mean absence of methylation of one or both of the 5-position on the pyrimidine rings located within the CpG motif. One or more CpG-motifs may be unmethylated within a vector that is resistant against methylation. The resistance against methylation is achieved by replacement of an existing cytosine with a cytosine analogue.

The term “receiver host cell” is intended to mean a cell that is different as compared to the donor host cell. Inside a receiver host cell, a vector becomes methylated at the CpG motifs since the receiver host cell initiates methylation of CpG motifs at nucleotide sequences that are different as compared to the nucleotide sequence of the receiver host cell. This may occur when the receiver host cell receives a vector that has been produced/multiplied in a donor host cell. The methylation of the vector results in a reduced or complete silencing of the vector.

The term “replaced with a cytosine analogue” is intended to mean an existing cytosine in a CpG motif as defined above being replaced by a cytosine analogue. The cytosine analogue replaces at least the 5-position of the pyrimidine ring of cytosine. The cytosine analogue preferably is resistant against methylation. However, the cytosine analogue may replace cytosine partly or completely. Cytosine is replaced with a cytosine analogue during replication of the vector.

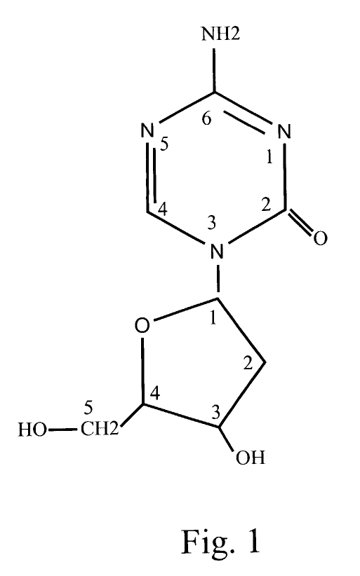

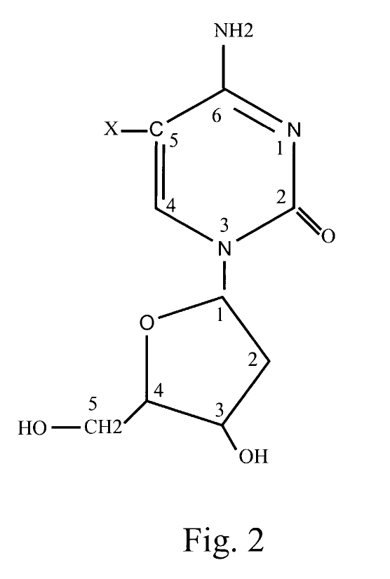

The term “cytidine derivative” is intended to mean a cytidine derivative able to replace cytosine in a CpG motif without influencing the activity of the nucleotide sequence harbouring the replaced cytidine derivative, e.g., the activity of the nucleotide sequence is the same as prior replacement of cytosine with a cytidine derivative. For example a nucleotide sequence encoding a polypeptide will, after replacement of one or more cytosines with cytidine derivatives, still be active and express the polypeptide. The cytidine derivative is a derivative that is unable to undergo methylation at the 5-position of the pyrimidine ring present in the cytidine derivative. Examples of cytidine derivatives are found in FIGS. 1 and 2.

The term “maintain expression” is intended to mean expression of the nucleotide sequence encoding a polypeptide and harbouring one or more cytosine analogues, i.e., maintain the expression to at least an acceptable level. One preferred level is higher than the unmethylated form of the nucleotide sequence. The maintained expression may be within a range that is higher than the expression of the methylated form of the same nucleotide sequence, or the expression may be as high as the expression of the unmethylated form of the same nucleotide sequence.

The term “donor host cell” is intended to mean a cell used for the production or multiplication of the vector. During growth or multiplication of the donor host cell, the copy number of the vector increases. At the same time, cytosine in CpG motifs is replaced with a cytidine analogue when the cytosine analogue is added in a sufficient amount to the growth medium used for growth of the donor host cell. The donor host cell is different as compared to the receiver host cell. For example, a donor host cell can be a bacterial cell or a viral particle, and the receiver host cell can be an animal or a plant cell.

The term “and/or” is intended to mean (1) both of the terms which are joined by “and/or” and (2) one or the other of the terms which are joined by “and/or.”

The term “or” is intended to mean (1) both of the terms which are joined by “and/or” and (2) one or the other of the terms which are joined by “and/or.”

### Vector

A vector according to the invention is a vector comprising a nucleotide sequence in which one or two cytosines in at least one CpG motif has been replaced with a cytosine analogue, the cytosine analogue being resistant against methylation.

By providing said vector, which is resistant against methylation, the expression of the nucleotide sequences located within the vector is maintained to a level that is higher as compared to the methylated form of the vector. As a result, the function of the vector is maintained. Furthermore, replacement of cytosine with cytosine analogues may be used to control the expression of nucleotide sequences. Reducing or increasing the level of methylation results in different levels of expression, such that an increased level of methylation results in a decrease of the level of expression. By replacing cytosine with a cytosine analogue, it is possible to avoid methylation problems that may arise when the vector is introduced into a receiver host cell.

According to a first preferred embodiment, cytosine is replaced with a cytosine analogue that replaces the 5-position in the pyrimidine ring of cytosine located in the CpG motif. The cytosine analogue may be N, O or C—X. X in C—X may be a low or non-electrophilic group, such as ethyl or methoxy. Furthermore the cytosine may be replaced by a cytidine derivative, such as 5-azacytidine and/or 5-azadeoxycytidine. 5-azadeoxycytidine may also be named s-Triazin-2(1H)-one, 4-amino-1-(2-deoxy-.beta.-D-erythro-pentofuranosyl- (8CI), 1,3, 5-Triazin-2(1H)-one, 4-amino-1-(2-deoxy-.beta.-D-erythro-pentofuranosyl)-(9CI), 5-Aza-2′-deoxycytidine or Decitabine (USAN).

The number and the distribution of the cytosine analogues within CpG motifs of the vector inhibits methylation of the 5-position of the pyrimidine ring resulting in maintenance of the expression of the nucleotide sequence encoding polypeptides located within the vector. An increased number of integrated cytosine analogues within a nucleotide sequence region encoding a polypeptide of the vector result in an increased possibility of maintaining the expression of the nucleotide sequences present in that region of nucleotide sequences. Increased number of integrated cytosine analogues within other regions of the vector may indirectly influence the maintenance of a nucleotide sequence region encoding a polypeptide. Examples of nucleotide sequence regions, which indirectly influence other nucleotide sequence regions, are control sequences, such as promoters.

A CpG motif includes two cytosines having the ability of being replaced by a cytosine analogue. Either one or both of them may be replaced. In the case where several cytosine analogues are integrated into the vector, the cytosine analogues may either be integrated in one and the same strand or both strands. The cytosine analogues are integrated into the vector during replication of that vector.

The number of cytosine analogues and the their distribution result in resistance against methylation of said cytosine analogues. The presence of said cytosine analogues maintain the expression of nucleotide sequences including cytosine analogues to an acceptable level.

In one embodiment, at least 1% (e.g. 1-5%), of the CpG motifs are replaced with cytosine analogues, such as 5% (e.g. 5-10%), 10% (e.g. 10-20%), 20% (e.g. 20-30%), 30% (e.g. 30-40%), 40% (e.g. 40-50%), 50% (e.g. 50-60%). Preferably from about 1 to about 100% of the cytosines are replaced by cytosine analogues, such as from I to about 95% or from about 5 to about 95%. The cytosine analogues are preferably integrated either into one or both strands of the nucleotide sequence of the vector.

The vector may comprise at least one gene or part of one gene. Which gene depends on what the vector will be used for. A potential gene candidate may be one that gives rise to an immune response against influenza and that is useful for the purpose of vaccination of human beings. Furthermore, the vector may comprise one or more control sequences. Such control sequences, for example, preferably enable the possibility to maintain and multiply the vector within a suitable donor host and preferably include control sequences that enable the possibility to maintain and/or multiply the vector within the receiver host cell. Additional control sequences may be present that enable the possibility to express a gene and/or part of a gene within a receiver host cell at a suitable level of expression. The choice of control sequences is dependent on the desired level of expression of the gene or part of the gene and a person skilled in the art will be able to identify such control sequences.

The vector may be a nucleotide sequence as defined above which may also be used therapeutically as anti-sense DNA. Concerns are taken in the production of such DNA and CpG di-nucleotides are omitted, if possible, in the constructs. Antisense DNA is used, for example, to interfere with the nuclear DNA and triple helix motifs are formed. Such triple helix structures are impaired in transcriptional activity or the interfering DNA can be coupled to strand-breaking activities. Anti sense DNA can also be used to bind mRNA in such a way so that the translation of the mRNA is impaired, RNase H activity is induced, and the mRNA is degraded.

The vector may be a nucleotide sequence as defined above, which may form aptameres. Aptameres are rather short linear DNA that binds in a specific manner to different biological molecules, such as specific protein motifs, carbohydrates, and steroids. The aptameres can thereby block the activity of certain biological molecules.

The vector may be any vector as long as the vector is capable of multiplying within a host (receiver and/or donor), i.e., having a start of replication. It should be understood that not all vectors and control sequences function equally well to express a nucleotide sequence of interest. Neither will a host function equally well with the same expression system. However, a person skilled in the art will be able to make a selection among these vectors, control sequences, and hosts without undue experimentation. For example, in selecting a vector, the host must be considered because the vector must replicate in the donor and/or the receiver host cell, or be able to integrate into the chromosome of the receiver host cell. The copy number of the vector, the ability to control the copy number, and the expression of any other proteins encoded by the vector, such as selectable markers, should also be considered.

In selecting a control sequence, a variety of factors should be considered. These include, for example, the relative strength of the sequence, its controllability, and its compatibility with the nucleotide sequence encoding the polypeptide of interest, particularly with regard to potential secondary structures. Hosts should be selected by consideration of their compatibility with the chosen vector, the toxicity of the polypeptide encoded by the nucleotide sequence, their secretion characteristics, their ability to fold the polypeptide correctly, their fermentation or culture requirements, and the ease of purification of the products encoded by the nucleotide sequence.

The vector may be an autonomously replicating vector, i.e., a vector that exists as an extra chromosomal entity, the replication of which is independent of chromosomal replication, e.g., a plasmid. Alternatively, the vector is one that integrates into the receiver host cell genome and replicates together with the chromosome(s) into which it has been integrated. A vector that integrates into the genome of the receiver host cell may be advantageous to use when a certain gene and/or part of a gene, and/or control sequence need to be protected against future possible methylation problems. Methylation might occur upon replication of the genome into which the vector has been integrated. Another example is when there is a need to remove a certain gene and/or part of a gene and/or control sequence present in a genome. The expression of the gene and/or part of the gene and/or the control sequence may be reduced due to methylation, and the replacement of such a methylated gene and/or part of a gene and/or control sequence with an unmethylated form may increase the pre-existing expression level. The replacement may be performed using methods such as homologous recombination using linear DNA. The gene and/or part of a gene and/or control sequence may, by such replacements, be up or down regulated. A vector comprising one or more cytidine analogues that is integrated into a genome may, upon replication of the genome, give rise to new copies of the genome comprising the vector without one or more cytidine analogues. However, the new copy of a genome harbouring a vector may anyway be resistant against methylation even if no cytidine analogue is present within the new copy of the vector located in the new copy of the genome.

The vector may be an expression vector in which the nucleotide sequence encoding the polypeptide of the invention is operably linked to additional segments required for transcription of the nucleotide sequence. The vector is typically derived from plasmid or viral DNA. A number of suitable expression vectors for expression in the host cells mentioned herein are commercially available or described in the literature. Useful expression vectors for eukaryotic hosts, include, for example, vectors comprising control sequences from SV40, bovine papilloma virus, adenovirus and cytomegalovirus. Specific vectors are, e.g., pCDNA3.1(+)Hyg (Invitrogen, Carlsbad, Calif., U.S.A.) and pCI-neo (Stratagene, La Jolla, Calif., U.S.A.). Useful expression vectors for yeast cells include the 2μ plasmid and derivatives thereof, the POT1 vector (U.S. Pat. No. 4,931,373), the pJSO37 vector described in Okkels, Ann. New York Acad. Sci. 782, 202-207, 1996, and pPICZ A, B or C (Invitrogen). Useful vectors for insect cells include pVL941, pBG311 (Cate et al., “Isolation of the Bovine and Human Genes for Mullerian Inhibiting Substance and Expression of the Human Gene in Animal Cells”, Cell, 45, pp. 685-98 (1986), pBluebac 4.5 and pMelbac (both available from Invitrogen). Useful expression vectors for bacterial hosts include known bacterial plasmids, such as plasmids from E. coli, including pBR 322, pET3a and pET12a (both from Novagen Inc., Wis., U.S.A.), wider host range plasmids, such as RP4, phage DNAs, e.g., the numerous derivatives of phage lambda, e.g., NM989, and other DNA phages, such as M13 and filamentous single stranded DNA phages. Examples of suitable viral vectors are Adenoviral vectors, Adeno associated viral vectors, retroviral vectors, lentiviral vectors, herpes vectors and cytomegalo viral vectors.

Other vectors for use in this invention include those that allow the nucleotide sequence encoding the polypeptide to be amplified in copy number. Such amplifiable vectors are well known in the art. They include, for example, vectors able to be amplified by DHFR amplification (see, e.g., Kaufman, U.S. Pat. No. 4,470,461, Kaufman and Sharp, “Construction Of A Modular Dihydrafolate Reductase cDNA Gene: Analysis Of Signals Utilized For Efficient Expression”, Mol. Cell. Biol., 2, pp. 1304-19 (1982)) and glutamine synthetase (“GS”) amplification (see, e.g., U.S. Pat. No. 5,122,464 and EP 338,841).

The recombinant vector may further comprise a DNA sequence enabling the vector to replicate in the host cell in question. An example of such a sequence (when the host cell is a mammalian cell) is the SV40 start of replication. When the host cell is a yeast cell, suitable sequences enabling the vector to replicate are the yeast plasmid 2μ replication genes REP 1-3 and start of replication.

The vector may also comprise a selectable marker, e.g., a gene, the product of which complements a toxin related deficiency in the host cell, such as the gene coding for dihydrofolate reductase (DHFR) or the Schizosaccharomyces pombe TPI gene (described by P. R. Russell, Gene 40, 1985, pp. 125-130), or one which confers resistance to a drug, e.g., ampicillin, kanamycin, tetracyclin, chloramphenicol, neomycin, hygromycin or methotrexate. For Saccharomyces cerevisiae, selectable markers include ura3 and leu2. For filamentous fungi, selectable markers include amdS, pyrG, arcB, niaD and sC.

The term “control sequences” is defined herein to include all components, which are necessary or advantageous for the expression of the polypeptide of the invention. Each control sequence may be native or foreign to the nucleic acid sequence encoding the polypeptide. Such control sequences include, but are not limited to, a leader sequence, a polyadenylation sequence, a propeptide sequence, a promoter (inducible or constitutive), an enhancer or upstream activating sequence, a signal peptide sequence, and a transcription terminator. At a minimum, the control sequences include a promoter.

A wide variety of control sequences may be used in the present invention. Such useful control sequences include the control sequences associated with structural genes of the foregoing expression vectors, as well as any sequence known to control the expression of genes of prokaryotic or eukaryotic cells or their viruses, and various combinations thereof.

Examples of suitable control sequences for directing transcription in mammalian cells include the early and late promoters of SV40 and adenovirus, e.g., the adenovirus 2 major late promoter, the MT-1 (metallothionein gene) promoter, the human or rodent cytomegalovirus immediate-early gene promoter (CMV), the human elongation factor 1α (EF-1α) promoter, the Drosophila minimal heat shock protein 70 promoter, the Rous Sarcoma Virus (RSV) promoter, the human ubiquitin C (UbC) promoter, the human growth hormone terminator, SV40 or adenovirus Elb region polyadenylation signals and the Kozak consensus sequence (Kozak, M. J Mol Biol 1987 Aug. 20;196(4):947-50).

In order to improve expression in mammalian cells, a synthetic intron may be inserted in the 5′ untranslated region of the nucleotide sequence encoding the polypeptide. An example of a synthetic intron is the synthetic intron from the plasmid pCI-Neo or from the β-globin gene (such as is available from Promega Corporation, Wis., U.S.A.). Moreover, an IRES element might be included.

Examples of suitable control sequences for directing transcription in insect cells include the polyhedrin promoter, the P 10 promoter, the Autographa californica polyhedrosis virus basic protein promoter, the baculovirus immediate early gene 1 promoter and the baculovirus 39K delayed-early gene promoter, and the SV40 polyadenylation sequence. Examples of suitable control sequences for use in yeast host cells include the promoters of the yeast α-mating system, the yeast triose phosphate isomerase (TPI) promoter, promoters from yeast glycolytic genes or alcohol dehydrogenase genes, the ADH2-4c promoter, and the inducible GAL promoter. Examples of suitable control sequences for use in filamentous fungal host cells include the ADH3 promoter and terminator, a promoter derived from the genes encoding Aspergillus oryzae TAKA amylase triose phosphate isomerase or alkaline protease, an A. niger α-amylase, A. niger or A. nidulans glucoamylase, A. nidulans acetamidase, Rhizomucor miehei aspartic proteinase or lipase, the TPI1 terminator and the ADH3 terminator. Examples of suitable control sequences for use in bacterial host cells include promoters of the lac system, the trp system, the TAC or TRC system, and the major promoter regions of phage lambda.

The presence or absence of a signal peptide will, for example, depend on the expression host cell used for the production of the polypeptide to be expressed (whether it is an intracellular or extracellular polypeptide) and whether it is desirable to obtain secretion. For use in filamentous fungi, the signal peptide may conveniently be derived from a gene encoding an Aspergillus sp. amylase or glucoamylase, a gene encoding a Rhizomucor miehei lipase or protease or a Humicola lanuginosa lipase. The signal peptide is preferably derived from a gene encoding A. oryzae TAKA amylase, A. niger neutral α-amylase, A. niger acid-stable amylase, or A. niger glucoamylase. For use in insect cells, the signal peptide may conveniently be derived from an insect gene (cf. PCT publication no. WO 90/05783), such as the Lepidopteran manduca sexta adipokinetic hormone precursor, (cf. U.S. Pat. No. 5,023,328), the honeybee melittin (Invitrogen), ecdysteroid UDPglucosyltransferase (egt) (Murphy et al., Protein Expression and Purification 4, 349-357 (1993) or human pancreatic lipase (hp1) (Methods in Enzymology 284, pp. 262-272, 1997). A preferred signal peptide for use in mammalian cells is that of hG-CSF or the murine Ig kappa light chain signal peptide (Coloma, M. (1992) J. Imm. Methods 152:89-104). For use in yeast cells suitable signal peptides have been found to be the α-factor signal peptide from S. cereviciae (cf. U.S. Pat. No. 4,870,008), a modified carboxypeptidase signal peptide (cf. L. A. Valls et al., Cell 48, 1987, pp. 887-897), the yeast BAR1 signal peptide (cf. PCT publication no. WO 87/02670), the yeast aspartic protease 3 (YAP3) signal peptide (cf. M. Egel-Mitani et al., Yeast 6, 1990, pp. 127-137), and the synthetic leader sequence TA57 (W098/32867).

Any suitable donor host cell may be used for the maintenance and production of the vector of the invention, such as an eukaryotic or prokaryotic cell, for example bacteria, fungi (including yeast), plant, insect, mammal, or other appropriate animal cells or cell lines, as well as transgenic animals or plants. The donor host cell may be a donor host cell belonging to a GMP (Good Manufacturing Practice) certified cell-line, such as a mammalian cell-line. Examples of bacterial donor host cells include gram positive bacteria such as strains of Bacillus, e.g. B. brevis or B. subtilis, Pseudomonas or Streptomyces, or gram negative bacteria, such as strains of E. coli. Examples of suitable filamentous fungal donor host cells include strains of Aspergillus, e.g. A. oryzae, A. niger, or A. nidulans, Fusarium or Trichoderma. Examples of suitable yeast donor host cells include strains of Saccharomyces, e.g. S. cerevisiae, Schizosaccharomyces, Klyveromyces, Pichia, such as P. pastoris or P. methanolica, Hansenula, such as H. Polymorpha or Yarrowia. Examples of suitable insect donor host cells include a Lepidoptora cell line, such as Spodoptera frugiperda (Sf9 or Sf21) or Trichoplusioa ni cells (High Five) (U.S. Pat. No. 5,077,214). Examples of suitable mammalian donor host cells include Chinese hamster ovary (CHO) cell lines, (e.g. CHO-K1; ATCC CCL-61), Green Monkey cell lines (COS) (e.g. COS 1 (ATCC CRL-1650), COS 7 (ATCC CRL-1651)); mouse cells (e.g. NS/O), Baby Hamster Kidney (BHK) cell lines (e.g. ATCC CRL-1632 or ATCC CCL-10), and human cells (e.g. HEK 293 (ATCC CRL-1573)), as well as plant cells in tissue culture. Additional suitable donor cell lines are known in the art and available from public depositories such as the American Type Culture Collection, Rockville, Md.

In a specific embodiment, the invention relates to a nucleotide sequence obtained from a donor host harbouring the nucleotide sequence being part of the vector. The vector and the donor host are a vector and a donor host as described above. The nucleotide sequence may be a transcriptional unit comprising a promoter, a nucleotide sequence, such as a gene or part of a gene, and polyadenylation site. The nucleotide sequence may be a gene and/or part of a gene and/or one or more control sequences, as mentioned above. One example of gene or genes of interest are genes encoding nuclear core proteins, such as the influenza nuclear core protein encoding genes, which may be used for vaccination. Another example is the gene encoding tyrosine hydroxylase, which may be useful for the treatment of Parkinson's disease. A third example is gene encoding factor X or VIII involved in the blood clotting complex or interferon gamma. Additionally, all kinds of genes useful in gene therapy, such as treatment of cancer, are gene candidates useful to be used according to the invention. One example, is thymidine kinase from Herpes Simplex Virus (HSV). The nucleotide sequence may be used as antisense. Antisense DNA is used, for example, to interfere with the nuclear DNA so that triple helix motifs are formed. Such triple helix structures are impaired in transcriptional activity, or the interfering DNA can be coupled to strand breaking activities. Anti sense DNA can also be used to bind mRNA in such a way so that the translation of the mRNA is impaired and so that RNase H activity is induced and the mRNA is degraded. Additionally, the nucleotide sequence may be used as aptameres, which are short stretches of nucleotide sequences that bind in a specific manner to different biological molecules, such as specific protein motifs, carbohydrates, steroids etc. The use of a cytosine base analogue, such as aza-deoxy-cytidine, that cannot be methylated, can lower the immunogenicity of unmethylated DNA and thereby reduce the immunostimulatory side effects. The vector and/or the nucleotide sequence, such as transcriptional units amplified by PCR, may be used to transfect cells. The vector may be linearized and, for example, prokaryotic parts can be left out by digestion with restriction enzymes. Such in vitro produced DNA can be used for, e.g., immunization or in gene therapy, ex vivo or in vivo.

In another embodiment, the invention relates to a polypeptide produced by the donor host cell with the polypeptide being encoded by a nucleotide sequence being part of the vector according to the invention and described above.

In another embodiment, the invention relates to a pharmaceutical composition comprising the above-described vector and/or the donor host and/or a nucleotide sequence obtained from the vector and/or a polypeptide and a pharmaceutically acceptable diluent, carrier, adjuvant, or excipient.

The vector, nucleotide sequence, polypeptide or a pharmaceutical composition as described above may be transferred into a receiver host cell by the use of a suitable transformation method, such as electroporation, microprojectile bombardment, or liposome-mediated delivery.

The vector, nucleotide sequence, polypeptide or a pharmaceutical composition as described above may be used in therapy and/or in diagnostic applications.

The vector, nucleotide sequence, polypeptide or a pharmaceutical composition as described above may be used for the manufacture of a medicament for use in therapy and/or in diagnostic.

### Method of Making Vector

One embodiment of the invention relates to a method that reduces methylation of CpG motifs in a vector in a receiver host, which method comprises replacing at least one cytosine in a CpG motif with a cytosine analogue, the vector being described above. The method may be a cultivation method or a method that amplifies the vector, such as by PCR.

According to another embodiment according to the invention, the method comprising the steps of providing a vector, transferring the vector into a donor host cell, growing the donor host cell harbouring the vector in a growth medium consisting of a suitable amount of cytosine analogues, and harvesting the multiplied vector comprising of incorporated cytosine analogues in one or two cytosines in at least one CpG motifs. The vector being a vector as described above and the donor host being a donor host as described above.

The vector and the donor host are chosen dependent upon the purpose of creating that specific vector with incorporated cytosine analogues and a person skilled in the art may perform that selection. The vector is transferred or introduced into the donor host using a suitable method dependent on which donor host has been selected. The introduction of a vector into a bacterial donor host cell may, for instance, be effected by protoplast transformation (see, e.g., Chang and Cohen, 1979, Molecular General Genetics 168: 111 -115), using competent cells (see, e.g., Young and Spizizin, 1961, Journal of Bacteriology 81: 823-829, or Dubnau and Davidoff-Abelson, 1971, Journal of Molecular Biology 56: 209-221), electroporation (see, e.g., Shigekawa and Dower, 1988, Biotechniques 6: 742-751), or conjugation (see, e.g., Koehler and Thorne, 1987, Journal of Bacteriology 169: 5771-5278). Fungal cells may be transformed by a process involving protoplast formation, transformation of the protoplasts, and regeneration of the cell wall in a manner known per se. Suitable procedures for transformation of Aspergillus host cells are described in EP 238 023 and U.S. Pat. No. 5,679,543. Suitable methods for transforming Fusarium species are described by Malardier et al., 1989, Gene 78: 147-156 and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153: 163; Hinnen et al., 1978, Proceedings of the National Academy of Sciences USA 75: 1920, and as disclosed by Clontech Laboratories, Inc, Palo Alto, Calif., U.S.A. (in the product protocol for the Yeastmaker™ Yeast Transformation System Kit). Transformation of insect cells and production of heterologous polypeptides therein may be performed as described by Invitrogen. Methods for introducing exogeneous DNA into mammalian donor host cells include calcium phosphate-mediated transfection, electroporation, DEAE-dextran mediated transfection, liposome-mediated transfection, viral vectors and the transfection method described by Life Technologies Ltd, Paisley, UK using Lipofectamin 2000. These methods are well known in the art and e.g., described by Ausbel et al. (eds.), 1996, Current Protocols in Molecular Biology, John Wiley & Sons, N.Y., U.S.A.

In the production methods of the present invention, the cells are cultivated in a growth medium suitable for maintenance and/or production of the vector using methods known in the art. For example, the cell may be cultivated by shake flask cultivation, small-scale or large-scale fermentation (including continuous, batch, fed-batch, or solid state fermentations) in laboratory or industrial fermentors performed in a suitable medium and under conditions allowing the vector, nucleotide sequence, or polypeptide to be expressed and/or isolated. The vector, nucleotide sequence, or polypeptide may be used in the chemical or pharmaceutical industries. The cultivation takes place in a suitable growth medium comprising carbon and nitrogen sources and inorganic salts, using procedures known in the art. Suitable media are available from commercial suppliers or may be prepared according to published compositions (e.g., in catalogues of the American Type Culture Collection). The cultivation of mammalian donor cells are conducted according to established methods, e.g., such as disclosed in one or more of the following references: Animal Cell Biotechnology, Methods and Protocols, Edited by Nigel Jenkins, 1999, Human Press Inc, Totowa, N.J., U.S.A. and Harrison M. A. and Rae I. F., General Techniques of Cell Culture, Cambridge University Press 1997.

The cytosine analogue, as described above, are added to the growth medium in a suitable amount sufficient to obtain a vector having a sufficient amount of cytosine analogues incorporated into the nucleotide sequence to yield a desired expression of nucleotide sequences encoding polypeptides. The amount of cytosine analogue is dependent on several factors including the size of the vector, the amount of CpG motifs, and the host cell. For example, in eukaryotic cells, 10 μM of 5-azacytidine and/or 5-azadeoxycytidine often is used. The cytosine analogue may be added either prior to cultivation or during the cultivation step.

After cultivation, the donor host is harvested by, for example, centrifugation and the vector is recovered by methods known in the art. Examples of methods are those mentioned in Maniatis et al., Molecular Cloning: A Laboratory Manual (Cold Spring Harbour Press) (1989) and Qiagen, Inc.

The amount and distribution of cytosine analogues present in the vector after production in a suitable donor host may be determined by, for example, using labelled 5-azacytidine and/or 5-azadeoxycytidine, HPLC, GC or TC.

Additionally, the vector, as defined above, may be produced by amplification, such as by the use of PCR. One example is the amplification of a transcriptional unit by PCR and the use of such units to transfect cells directly. Another possibility is to use the vector of the invention, such as a plasmid vector, for transfection. The vector may be linearized and, e.g., prokaryotic parts can be left out by digestion with restriction enzymes. By the use of PCR, all sites that may be replaced by a cytosine analogue can be replaced due to the absence of dCTP during the amplification reaction. The amplified vector may be used for immunization or in gene therapy, ex vivo or in vivo. One example is transfection of a mammalian cell line with a transcriptional unit, including a promoter, a cDNA and a polyadenylation site, which may be done to produce a protein encoded by the cDNA that will be secreted from the cells in a fermentor.

### Kit

Accordingly, the invention relates to a kit comprising a vector, as described above, and/or a donor host cell, as described above, including a description how to use the vector and/or the vector and the donor host cell.

The kit may further comprise a growth medium suitable for the donor host cell for the ability to grow (multiply) the vector.

The kit may be used for the production of a polypeptide in industry or in a method for the treatment of a mammal suffering from a disorder or in need of vaccination. Examples of disorders are influenza and cancer.

### Pharmaceutical Use and Formulations

According to one embodiment of the invention the vector, donor host, nucleotide sequence, polypeptide, or the pharmaceutical composition, according to the invention, may be used for several applications, including vaccination and gene therapy.

However, when the vector and/or the donor host is used for the treatment of a disorder or vaccination, it is important that the nucleotide sequences creating the vector are nucleotide sequences (e.g., start of replication and control sequences) that maintain their activity, such as express a polypeptide within the receiver host, i.e. the cells of the patient. For example, in the case when the vector is used for vaccination of a human, it is important that a promoter is used upstream of the nucleotide sequence encoding a polypeptide that gives rise to the immune response, i.e., the promoter is active in a human cell. The promoter may be inducible and/or constitutive. In the case of using an inducible promoter, it may be inducible by an agent either administered together or subsequently with the vector and/or the donor host. An agent produced within the patient, either locally or systemically, may further induce the promoter.

For example, if plasmids encoding insulin is introduced intramuscularly by electroporation, then this plasmid will express insulin for a while and thereafter the expression diminishes. A methylation resistant plasmid, on the other hand, will probably have a constant expression for a long period of time. If such a plasmid has a tetracycline-inducible promoter, the insulin production could be controlled by oral administration of tetracycline. This type of gene therapy could be applicable for patients with insulin deficient type diabetes, or if the gene to be expressed is a missing coagulation factor by hemophilic patients.

In a second embodiment, the vector/donor host, nucleotide sequence, polypeptide, or the pharmaceutical composition according to the invention is used for the manufacture of a medicament for treatment of disorders or vaccination, such as cancer. In cancer therapy of today there is a focus on therapies other than the classical radiotherapy and chemotherapy. Anti-angiogenic therapy, as well as immunotherapy against cancer are examples of two promising fields. Both therapies can utilise gene therapy for the production of anti-angiogenic agents, or for the production of immunostimulatory or toxic agents. These types of therapy can be optimised by the use methylation resistant vectors, both as pure plasmids, or as viral vectors made resistant against methylation. For example, a genetically modified adenovirus which expresses the gene for Herpes Simplex Thymidine kinase (HSV-TK) may be produced in the presence of a described cytosine analogue. Then, the DNA of the adenovirus will contain this analogue and, upon infection, the adenovirus will express higher levels of HSV-TK for a longer period of time as compared to an adenovirus not having methylation resistant DNA. The tumor cells can then be killed by a herpes virus drug. The killing is correlated with the production of HSV-TK, i.e., the higher the concentration of HSV-TK, the more sensitive the tumor cell will be for the drug.

In another embodiment, the vector/donor host, nucleotide sequence, polypeptide or the pharmaceutical composition, according to the invention, is used in a method for treating a mammal having a disorder or in need of vaccination. Vaccination has traditionally been performed by the use of mutated or killed pathogenic viruses. DNA vaccination makes use of proteins encoded by the DNA of the pathogen. Such a gene or genes are cloned in a plasmid or another vector. Plasmids encoding pathogenic protein(s) can be injected, e.g., intramuscularly. The plasmid will express the pathogenic protein(s) that are recognised by the immune system of that individual. This type of immunisation then protects against subsequent infection of the pathogen. The strength of the immune reaction depends on the production of the pathogenic protein. A too low production of the pathogenic protein(s) might not be recognised by the immune system. Hence, immunisation of both humans and animals with methylation resistant vectors will improve the efficiency.

Pharmaceutical formulations of the vector/donor host, nucleotide sequence, and/or polypeptide of the invention are typically administered in a composition that includes one or more pharmaceutically acceptable carriers or excipients. Such pharmaceutical compositions may be prepared in a manner known in the art that is sufficiently storage stable and suitable for administration to humans and animals. The pharmaceutical composition may be lyophilised.

“Pharmaceutically acceptable” means a carrier or excipient that, at the dosage and concentrations employed, does not cause any unwanted effects in the patients to whom it is administered. Such pharmaceutically acceptable carriers or excipients are well known in the art. For example, see Remington's Pharmaceutical Sciences, 18th edition, A. R Gennaro, Ed., Mack Publishing Company (1990) and handbook of Pharmaceutical Excipients, 3rd edition, A. Kibbe, Ed ., Pharmaceutical Press (2000).

The pharmaceutical composition may be admixed with adjuvants, such as lactose, sucrose, starch powder, cellulose esters of alkanoic acids, stearic acid, talc, magnesium stearate, magnesium oxide, sodium, and calcium salts of phosphoric and sulphuric acids, acacia, gelatin, sodium alginate, polyvinyl-pyrrolidine, and/or polyvinyl alcohol, and tableted or encapsulated for conventional administration. Alternatively, they may be dissolved in saline, water, polyethylene glycol, propylene glycol, ethanol, oils, such as corn oil, peanut oil, cottonseed oil or sesame oil, tragacanth gum, and/or various buffers. Other adjuvants and modes of administration are well known in the pharmaceutical art. The carrier or diluent may include time delay material, such as glyceryl monostearate or glyceryl distearate, alone or with a wax, or other materials well known in the art.

The pharmaceutical compositions may be subjected to conventional pharmaceutical operations, such as sterilisation, and/or may contain conventional adjuvants, such as preservatives, stabilisers, wetting agents, emulsifiers, buffers, fillers, etc., e.g., as disclosed elsewhere herein.

The pharmaceutical composition according to the invention may be administered locally or systemically, such as topically, intravenously, orally, parenterally or as implants, and even rectal use is possible. Suitable solid or liquid pharmaceutical preparation forms are, for example, granules, powders, tablets, coated tablets, capsules, such as microcapsules, suppositories, syrups, emulsions, suspensions, creams, aerosols, drops, or injectable solution in ampule form, and also preparations with protracted release of active compounds, in whose preparation excipients, diluents, adjuvants, or carriers are customarily used as described above.

The pharmaceutical composition will be administered to a patient in a pharmaceutically effective dose. By “pharmaceutically effective dose,” it is meant a dose that is sufficient to produce the desired effects in relation to the condition for which it is administered. The exact dose is dependent on the activity of the compound, the manner of administration, the nature and severity of the disorder, the age and body weight of the patient because different doses may be needed. The administration of the dose can be carried out both by single administration in the form of an individual dose unit, several smaller dose units, and by multiple administrations of subdivided doses at specific intervals. Vaccination is one such example.

The pharmaceutical composition of the invention may be administered alone or in combination with other therapeutic agents. These agents may be incorporated as part of the same pharmaceutical composition or may be administered separately.

A “patient,” for the purposes of the present invention, includes both humans and other mammals. Thus, the methods are applicable to both human therapy and veterinary applications.

Following examples are intended to illustrate but not to limit the invention in any manner, shape, or form, either explicitly or implicitly.

### EXAMPLES

### Example 1

### Production of Methylation Resistant Plasmid DNA in Bacteria

1. Inoculate 2 ml conventional LB medium and 50 microgram/ml of ampicillin in a 10 ml test tube with an E. coli harbouring a plasmid of interest.

2. The culture is incubated in a shaker (200 rpm) over night at 37° C.

3. The over night culture is then used to inoculate a 500 ml LB medium in a 2 litre culture flask. The flask is incubated at 37° C. in a shaker until the growth of the bacteria reaches OD (650) of 0.5 to 0.8.

4. 5′-deoxy-azacytidine or another deoxy-cytidine analogue is added to a concentration of 10 μM and chloramphenicol to a final concentration of 180 μg/ml is added.

5. The culture is incubated at 37° C. on a shaker as above overnight.

6. The bacteria are pelleted by conventional centrifugation and plasmid DNA is isolated according to a standard Qiagen Inc., Maxiprep protocol.

### Example 2

### Production of Adenovirus with Methylation Resistant DNA

1. Plate 911 or 293 cells in conventional T-75 flasks to be 60-90% confluent at time of infection (about 1×10⁷ cells/T-75). Usually, fifteen to twenty T-75 flasks are sufficient to make a high titer stock.

2. Infect cells with virus supernatant at a multiplicity of infection (MOI) of 5 to 10 PFU (plaque forming units) per cell.

3. At day 2, add 5′-deoxy-azacytidine to a concentration of 10 μM.

4. When all cells have rounded up and about half of the cells are detached (usually at 3 to 4 days post infection), harvest and combine all flasks. Spin 5 minutes in a bench top centrifuge (˜500 g), and remove the supernatant.

5. Resuspend pellet in 8.0 ml sterile PBS. Perform four cycles of freeze/thaw/vortex. Centrifuge lysate in Sorvall HS4 rotor at 6000 rpm (7000 g) 4° C. for 5 min.

6. Weigh 4.4 grams of CsCl in a 50-ml conical tube, transfer 8.0 ml of clear virus supernatant to the tube, and mix well by vortexing. Transfer the CsCl solution (about 10 ml, density of 1.35 g/ml) to a 12 ml polyallomer tube for SW41 rotor. Overlay with 2 ml mineral oil. Prepare a balance tube. Spin the gradient in SW41 rotor at 32,000 rpm, 10° C., for 18 to 24 hours.

7. Collect virus fraction (about 0.5 to 1.0 ml) with a 3 cc syringe and an 18 g needle. Insert needle through the tube from below. Mix with equal volume 2X Storage Buffer (2X Storage Buffer=10 mM Tris, pH 8.0, 100 mM NaCl, 0.1% BSA, and 50% glycerol, filter sterilized). Store virus stocks at −20° C.

8. Check viral titer by GFP (preferred) of by plaque assays (see below) or by immunohistochemical staining, or simply read OD at 260 nm. To read OD, add 15 μl virus to 15 μl blank solution (Blank Solution=1.35 g/ml CsCl mixed with equal vol 2X Storage Buffer) plus 100 μl TE/0.1% SDS; vortex 30 seconds, centrifuge 5 min. measure A260. One A260 unit contains −1×1012 viral particles (particles: infectious particles should be about 20:1).

### Example 3

### Measurement of the Degree of 5-azadeoxycytidine Incorporation in DNA

### Method 1

Add 5′-deoxy-azacytidine to the media of the growing cells at a concentration of 10 μM. Also add a trace amount ¹⁴C-labelled deoxy-azacytidine, e.g., 5 μCi. If the specific activity of the labelled azacytidine is 500 Ci/mmol this is equivalent to 10 pmol of azacytidine. If the volume used is 100 ml, then the concentration of the radio-labelled nucleotide is 100 pM. The relation between the cold and labelled nucleotide is then 100.000:1. The radioactivity of the isolated DNA is measured in a scintillation counter and the incorporation is estimated dependant on the efficiency of the scintillation counter (1 Ci =1×10¹⁰ dps).

### Method 2

Add 5′-deoxy-azacytidine to the media of the growing cells at a concentration of 10 μM. The aza-cytdine molecule can be separated by means of HPLC from cytidine. The isolated DNA is treated with DNAse I so that a high concentration of free nucleotides is generated. The samples are injected into a suitable HPLC column and the nucleotides are respectively separated into cytidine, thymidine, guanine, adenine and 5′ azacytdine. The concentration of aza-cytidine in relation to cytidine is measured.

### Example 4

### Production of Vector DNA, In Vitro, using PCR

A transcriptional unit containing a CMV promoter, the gene encoding green fluorescent protein (GFP) and an HSV-TK polyadenylation site framed by two insulators in the opposite direction is cloned in a plasmid. The opposite directions of the two chicken beta globin insulators are used so that if recombination occurs, the orientation of the transcriptional unit is only inverted and not lost. The purpose is to amplify the transcriptional unit with base analogues that make it methylation resistant and to introduce the unit into human glioma cells. Different ratios between the base analogue 5′-aza-2′-deoxy cytidine triphosphate and 2′-deoxy cytidine triphosphate are used to generate vector DNA with different levels of methylation resistant 5′-aza cytidine.

1. 10 ng of the cloned gene construct in the shuttle vector pAd-easy is mixed in a reaction tube containing a buffer for pfu DNA polymerase with 5 mM of MgCl₂, 100 mM Tris at pH 7,4, deoxy-nucleotide triphosphate mixture containing 250 micromolar of dATP, dGTP and dTTP. The reaction mixture also contains a mixture between 5′-aza-2-deoxycytidine triphosphate and 2′-deoxycytidine triphosphate in the following ratios; 100:0, 75:25, 50:50, 25:75, and 0:100 so that the final concentration is 250 micromolar of the two cytidines together. The reaction mixture also contains two primers, forward primer is CAGGACTCTGATGGAACCAGG (SEQ. ID. NO: 1) and the reverse primer is CCCTACCATTAGATGGATCAG (SEQ. ID. NO: 2). The reaction mixture contains 250 ng of each primer and the reaction volume is 50 microliters.

2. To the five different tubes, 2 units of pfu DNA polymerase (Stratagene Inc., U.S.A.) is added and 20 cycles of PCR are run with an annealing temperature of 56° C. for 1 minute, a reaction temperature of 72° C. for 2 minutes, and a denaturation temperature of 95° C. for 40 seconds.

3. After PCR, agarose gel electrophoresis under mild UV is performed on the five reactions, the five different bands are excised from the gel, and the DNA of the five different fragments is isolated from the gel.

4. For three different primary human glioma cell cultures, I microgram of each of the five different vectors are used for transfection following the protocol from Boehringer & Mannheim using FUGENE. The cell number that is used for each transfection is 5×10⁶ cells in ten different tissue culture flasks.

5. After 24 hours the cells are trypsinated and transferred to 10 new tissue culture flasks and 1×10⁵ cells are used for FACS analysis, monitoring the transient expression from the ten different samples.

6. One week after transfection, the cells are trypsinated again and the cells are sorted using the FACS (Becton Dickinson Inc., U.S.A.). The GFP positive cells are replated onto 24 well plates. The cultures are to be followed and monitored using a fluorescent inverted microscope (Olympus). Another two weeks after expanding the cultures, the cells are once again subjected to FACS cell sorting.

7. The GFP positive cells are diluted to 0.5 cells per 100 microliter and 100 microliter are put into 96 replicate wells for each of the ten different cultures. One-cell clones that originated from this cloning are measured for long-term expression of GFP.

### Example 5

### Construction of a Transgenic Plant

Competent cells of Agrobacterium tumefaciens are subjected to transfection with the plasmid pRT 101 in which the gene for green fluorescent protein are cloned in the Eco RI site of the plasmid and is under the control of the Cauliflower mosaic virus promoter (CaMV). The bacteria are grown in YM broth (0.04% yeast extract, 1% mannitol, 1.7 mM NaCl, 0.8 mM MgSO4.7H2O, 2.2 mM K2HPO4.3H2O, (pH 7.0)) and at an OD of 0.1, the medium was supplemented with 100 micromolar of 5′-aza-2′-deoxycytidine. The plasmid DNA is isolated using standard technique with a kit from Qiagene Inc.

The plasmid DNA isolated is used for electroporation of callus cells from barley (Hordeum vulgare). As a control, the same plasmid not containing aza-cytidine is used.

The transformation frequency is followed under an inverted fluorescent microscope and stable clones from both natural plasmid transfection as well as those transformed with plasmids containing aza-cytidine are subjected to differentiation and plants are developed from both categories. The expression of the jellyfish protein (GFP) over long term in the two groups is studied.

It is also to be understood that, although the foregoing description and drawings describe and illustrate in detail preferred embodiments of the present invention, to those skilled in the art to which the present invention relates, the present disclosure will suggest many modifications and constructions as well as widely differing embodiments and applications without thereby departing from the spirit and scope of the invention. The present invention, therefore, is intended to be limited only by the scope of the appended claims and the applicable prior art.

What is claimed is:

1. A vector comprising a nucleotide sequence wherein one or two cytosines in at least one CpG motif has been replaced with a cytosine analogue resistant to methylation.

2. The vector according to claim 1, wherein the cytosine analogue replaces the 5-position in the pyrimidine ring of cytosine located in the CpG motif with N, O or C—X.

3. The vector according to claim 2, wherein cytosine analogue comprises a cytidine derivative.

4. The vector according to claim 3, wherein the cytidine derivative comprises 5-azacytidine or 5-azadeoxycytidine.

5. The vector according to claim 2, wherein X is of a low or non-electrophilic group.

6. The vector according to claim 5, wherein the low non-electrophilic group is selected from the group consisting of ethyl and methoxy.

7. The vector according to claim 1, wherein the replaced cytosine is located in one and the same DNA strand or in both DNA strands of the vector.

8. The vector according to claim 1, wherein the vector comprises a linear vector, a circular vector, a single stranded vector, or a double stranded vector.

9. The vector according to claim 1, wherein the vector is selected from the group consisting of bacteriophages, plasmids, phagemids, viral vectors, plant transformation vectors, insect vectors, and yeast artificial chromosomes.

10. The vector according to claim 1, wherein the number of cytosine analogues and the distribution of the cytosine analogues render the nucleotide sequences, including the cytosine analogues, resistant against methylation and maintain the expression of the nucleotide sequences including cytosine analogue containing CpG motifs.

11. The vector according to claim 1, wherein at least 1% of the CpG motifs are replaced with cytosine analogues.

12. The vector according to claim 1, wherein at least 5% of the CpG motifs are replaced with cytosine analogues.

13. The vector according to claim 1, wherein at least 10% of the CpG motifs are replaced with cytosine analogues.

14. The vector according to claim 1, wherein at least from about 1% to about 100% of the CpG motifs are replaced with a cytosine analogue.

15. The vector according to claim 1, wherein the vector comprises a gene or a part of a gene.

16. The vector according to claim 1, wherein the vector comprises one or more expression control sequences.

17. A donor host cell comprising a vector that includes a nucleotide sequence, wherein one or two cytosines in at least one CpG motif has been replaced with a cytosine analogue resistant to methylation.

18. The donor host cell according to claim 17, wherein the donor cell is a eukaryotic or a procaryotic cell.

19. The donor host cell according to claim 18, wherein the donor host cell is selected from the group consisting of an isolated bacteria cells, fungi cells, plant cells, insect cells, animal cells, bacteria cell lines, fungi cell lines, plant cell lines, insect cell lines, animal cell lines, transgenic animals, and plants.

20. The donor host cell according to claim 19, wherein the donor host cell is a host cell belonging to a GMP certified cell-line.

21. The donor host cell according to claim 20, wherein the cell-line is a mammalian cell-line.

22. A nucleotide sequence obtained from a donor host cell that includes a vector comprising a nucleotide sequence, wherein one or two cytosines in at least one CpG motif has been replaced with a cytosine analogue resistant to methylation, and wherein the nucleotide sequence is part of the vector that includes said nucleotide sequence.

23. The nucleotide sequence according to claim 22, wherein the nucleotide sequence comprises a gene, a part of a gene, an expression control sequence, or a transcriptional unit.

24. A polypeptide produced by a donor host cell comprising a vector that includes a nucleotide sequence, wherein one or two cytosines in at least one CpG motif has been replaced with a cytosine analogue resistant to methylation, wherein the nucleotide sequence encoding the polypeptide is part of the vector.

25. A pharmaceutical composition comprising:
a vector that includes a nucleotide sequence, wherein one or two cytosines in at least one CpG motif has been replaced with a cytosine analogue resistant to methylation, or
a donor host cell that includes the vector, or
a nucleotide sequence obtained from the donor host cell, and
wherein the nucleotide sequence comprises part of the vector.

26. A method of reducing methylation of CpG motifs in a vector in a receiver host, said method comprising replacing at least one cytosine in a CpG motif with a cytosine analogue resistant to methylation.

27. The method according to claim 27, wherein the method comprises the steps of i) providing a vector; ii) transferring the vector into a donor host cell; iii) growing the donor host cell by harbouring the vector in a growth medium consisting of a suitable amount of cytosine analogues; and iv) harvesting the multiplied vector that includes incorporated cytosine analogues in at least one CpG motif.

28. The method according to claim 27, wherein the cytosine analogue comprises a cytidine derivative.

29. The method according to claim 28, wherein the cytidine derivative comprises 5-azacytidine or 5-azadeoxycytidine.

30. The method according to claim 27, wherein the harvested vector includes a nucleotide sequence, wherein one or two cytosines in at least one CpG motif has been replaced with a cytosine analogue resistant to methylation.

31. The method according to claim 26, wherein the donor host cell that includes a vector that includes a nucleotide sequence, wherein one or two cytosines in at least one CpG motif has been replaced with a cytosine analogue resistant to methylation.

32. A kit comprising:
a vector that includes a nucleotide sequence, wherein one or two cytosines in at least one CpG motif has been replaced with a cytosine analogue resistant to methylation; or
a host cell that includes a vector.

33. A method comprising:
transferring into a receiver host cell:
a vector that includes a nucleotide sequence, wherein one or two cytosines in at least one CpG motif has been replaced with a cytosine analogue resistant to methylation, wherein the nucleotide sequence is part of the vector; or
a nucleotide sequence obtained from a donor host cell that includes the vector, wherein the nucleotide sequence is part of the vector;
a polypeptide produced by the donor host cell; or
a pharmaceutical composition including the vector; and
wherein the method is selected from the group consisting of electroporation, microprojectile bombardment, and liposome mediated delivery.

34. A method of therapy, the method comprising administering to an animal in need of the therapy:
a vector that includes a nucleotide sequence, wherein one or two cytosines in at least one CpG motif has been replaced with a cytosine analogue resistant to methylation, or
a donor host cell that includes the vector, or
a nucleotide sequence obtained from the donor host cell, or
wherein the nucleotide sequence is part of the vector, or
a polypeptide produced by the donor host cell, or
a pharmaceutical composition including the vector.

35. A diagnostic method comprising:
adding to a sample being analysed to make a diagnoses:
a vector that includes a nucleotide sequence, wherein one or two cytosines in at least one CpG motif has been replaced with a cytosine analogue resistant to methylation, or
a donor host cell that includes the vector, or
a nucleotide sequence obtained from the donor host cell, or
wherein the nucleotide sequence is part of the vector, or a polypeptide produced by the donor host cell, or
a pharmaceutical composition including the vector.

36. A method of manufacturing a medicament, the method comprising producing the medicament from:
a vector that includes a nucleotide sequence, wherein one or two cytosines in at least one CpG motif has been replaced with a cytosine analogue resistant to methylation, or
a donor host cell that includes the vector, or
a nucleotide sequence obtained from the donor host cell,
wherein the nucleotide sequence is part of the vector, or
a polypeptide produced by the donor host cell, or
a pharmaceutical composition including the vector.


SEQUENCE LISTING (2 sequences)

SEQ ID NO: 1 - DNA, 21 bp, Artificial Sequence
  PCR primer
caggactctgatggaaccagg

SEQ ID NO: 2 - DNA, 21 bp, Artificial Sequence
  PCR primer
ccctaccattagatggatcag